(19) United States
(12) Patent Application Publication
(10) Pub. No.: US 2004/0248181 A1
(43) Pub. Date: Dec. 9, 2004

(54) Method and kit for enhancing extraction and quantification of target molecules using microdialysis

(76) Inventors: Julie A. Stenken, Troy, NY (US); Timothy J. Sellati, Glenmont, NY (US)

Correspondence Address: Michael L. Goldman, Nixon Peabody LLP, Clinton Square, P.O. Box 31051, Rochester, NY, 14603-1051, US

(21) Appl. No.: 10/858,219

(22) Filed: Jun. 1, 2004

Related U.S. Application Data
(60) Provisional application No. 60/475,418, filed on Jun. 3, 2003.

(51) Int. Cl. C12Q 1/68; G01N 33/53; G01N 33/543

(52) U.S. Cl. 435/6; 435/7.1

(57) ABSTRACT

The present invention is directed to a method for detection of a presence of one or more target molecules in a sample. This method involves providing a microdialysis unit having a membrane which defines within it a dialysate-containing region and contacting the microdialysis unit with a sample, potentially containing one or more target molecules, under conditions effective to permit molecules in the sample to permeate the membrane and enter the dialysate-containing region as a dialysate. A perfusate liquid comprising capture agents which are specific to the one or more target molecules and are immobilized to solid structures is provided and contacted with the dialysate (desirably within the dialysate-containing region) under conditions effective to permit target molecules present in the dialysate, if any, to specifically bind to the capture agents immobilized to the solid structures. As a result, a dialysate liquid potentially comprising dialysate complexes of target molecules bound to capture agents, which are immobilized to solid structures, is formed. The dialysate liquid is analyzed for dialysate complexes. As a result, the presence of the one or more target molecules in the sample is detected. A kit for carrying out this method is also disclosed.

The present application claims the benefit of U.S. Provisional Patent Application Ser. No. 60/475,418, filed Jun. 3, 2003.

### FIELD OF THE INVENTION

The present invention is directed to a method and kit for enhancing extraction and quantification of target molecules using microdialysis.

### BACKGROUND OF THE INVENTION

Microdialysis is a widely-used minimally invasive sampling technology that has been applied to animal and human in vivo chemical sampling applications (Benveniste et al., “Determination of Brain Interstitial Concentrations by Microdialysis,” J. Neurochem. 52:1741-50 (1989); Davies, M. I., “A Review of Microdialysis Sampling for Pharmacokinetic Applications,” Analytica Chimica Acta. 379:227-49 (1999); and Ungerstedt, U. “Microdialysis-Principles and Applications for Studies in Animals and Man,” J. Intern. Med. 230-365-373 (1991)). This technique is a well-established method for neurochemical applications (Robinson T. and Justice J. B. eds, Microdialysis in the Neurosciences Elsevier: Amsterdam), and is used more frequently in pharmacokinetic and metabolism studies (Hansen et al., “Pharmacokinetic and Metabolism Studies Using Microdialysis Sampling,” J. Pharm. Sci. 88:14-27 (1999)). In animal studies, microdialysis sampling meets the requirements of the “3Rs” for guiding animal research (refine, replace, and reduce), because samples from one individual animal can be obtained over multiple time points. This greatly reduces the number of experimental animals and allows the animal to serve as its own control when performing statistical analyses of the obtained microdialysis data.

A microdialysis probe consists of a piece of semi-permeable dialysis membrane (o.d. 250 to 600 μm; length 1 to 30 mm) with inserted inlet and outlet tubing. The microdialysis probe is perfused with an isotonic saline solution at flow rates typically between 0.5 and 5.0 μl/min. Solutes with molecular weight and size low enough to pass through the pores of the semi-permeable membrane will freely diffuse from the external sample space into the perfusion fluid. Because of the semi-permeable membrane, dialysis samples can be readily analyzed without further sample clean-up. When coupled to the appropriate analytical technology (sensor array, high performance liquid chromatography (“HPLC”), capillary electrophoresis, etc.), microdialysis sampling could be considered to be highly non-selective universal ‘analytical sensing’ device (Ballerstadt et al., “Sensor Methods for Use with Microdialysis and Ultrafiltration,” Adv. Drug Deliv. Rev. 21:225-38 (1996)).

Microdialysis sampling is a diffusion-based separation technique that, as currently practiced, has cylindrical geometry. Microdialysis is a diffusion-based process, and there are three regions of mass transport (dialysate perfusion fluid, membrane, and tissue) that must be accounted for during sampling. The fluid dynamics through the probe coupled with kinetic processes in the tissue complicate the coupling of the underlying diffusion equations thus making mathematical descriptions of diffusion discussed in classic texts (Crank and Cussler) unsuitable for microdialysis sampling (Crank, J., The Mathematics of Diffusion, Cambridge University Press, Cambridge (1975); Cussler, E. L., Diffusion: Mass Transfer in Fluid Systems, Cambridge University Press, Cambridge (1984)). By taking into account these coupled regions of mass transport, Bungay et al. have published a steady-state theory of microdialysis calibration (Bungay et al., “Steady-State Theory for Quantitative Microdialysis of Solutes and Water In vivo and In vitro,” Life Sciences 46:105-19 (1990)). The extraction efficiency (E_{d}), also sometimes referred to as relative recovery, shown in Equation (1), allows for calibration of microdialysis probes under steady-state conditions, where Cᵢₙₗₑₜ is the analyte inlet concentration, Cₒᵤₜₗₑₜ is the analyte outlet concentration, and C_{sample,∞}is the sample concentration far away from the probe (Bungay et al., “Steady-State Theory for Quantitative Microdialysis of Solutes and Water In vivo and In vitro,” Life Sciences 46:105-19 (1990)).

$${E}_{d} = \frac{{C}_{outlet} - {C}_{inlet}}{{C}_{sample , ∞} - {C}_{inlet}} \qquad ( 1 )$$

Measured dialysate concentrations of the analyte can be converted to analyte tissue concentrations by using the extraction efficiency (E_{d}) or calibration factor shown in Equation (1). E_{d} is influenced by any combination of the following experimental parameters: dialysis perfusion fluid flow rate and membrane length (Zhao et al., “Comparison of Recovery and Delivery In Vitro for Calibration of Microdialysis Probes,” Analytica Chimica Acta 316:403-10 (1995)), sample convection (Stenken et al., “Examination of Microdialysis Sampling in a Well-Characterized Hydrodynamic System,” Analytical Chemistry 65:2324-28 (1993)), and tissue diffusive and kinetic properties (Bungay et al., “Steady-State Theory for Quantitative Microdialysis of Solutes and Water In vivo and In vitro,” Life Sciences 46:105-19 (1990); Stenken et al., “Factors That Influence Microdialysis Recovery. Comparison of Experimental and Theoretical Microdialysis Recoveries in Rat Liver,” J. Pharm. Sci. 86:958-66 (1997)).

Conventional microdialysis sampling has revolutionized neurochemistry through its ability to obtain in vivo protein-free samples that contain small hydrophilic analytes such as dopamine and glucose from human and rodent mammalian brain (Ungerstedt, U., “Microdialysis-Principles and Applications for Studies in Animals and Man,” J. Intern. Med. 230:365-73 (1991); Robinson T. and Justice J. B. (eds), Microdialysis in the Neurosciences, Elsevier, Amsterdam (1991)). However, this sampling technique as currently practiced is not suitable for in vivo detection of larger biomolecules such as peptides and proteins. Microdialysis sampling is a dynamic technique and equal analyte concentrations between the perfusion fluid and external sample are rarely achieved. Large biomolecules have inherently small aqueous solution diffusion coefficients and exhibit hindered transport through the small pores of the dialysis membrane. This causes very low recovery values to be obtained due to mass transport limitations (Stenken, J.A., “Methods and Issues in Microdialysis Calibration,” Analytica Chimica Acta 379:337-58 (1999) and Snyder, et al., “Diffusion and Calibration Properties of Microdialysis Sampling Membranes in Aqueous and Protein Solutions,” Analyst 126:126-68 (2001)). Despite the use of membranes with molecular weight cutoff (MWCO) values of 100,000 Daltons (Da) or greater, recovery values of less than 2% are often observed for proteins in the range of 10,000 to 50,000 Da. This problem, coupled with the low in vivo concentrations of these analytes external to the device, causes great difficulty with identification and quantitation of larger biomolecules. These severe limitations preclude in vivo studies of numerous biomolecules at their site of action.

There is a great interest in improving the mass transport of different molecules through microdialysis sampling membranes. There have been different approaches described for enhancing relative recovery of lipophilic analytes. Stahle and Carneheim described approaches for improving oleic acid relative recovery in vitro using albumin (Cameheim et al., Pharmacol. Toxicol. 69:378-80 (1991)). By including albumin in the perfusate, the relative recovery (“RR”) of oleic acid was nearly doubled compared to controls. Others have included albumin in the perfusate to prevent non-specific adsorption of different analytes (Muller et al., J. Control. Rel. 37:49-57 (1995)). The difficulty with albumin addition to the microdialysis perfusion fluid is that it adds back to the microdialysate what microdialysis sampling was originally intended to prevent, principally large macromolecules. Generally, the assays used for measurements of such analytes are based on immunoassays that are tolerant to the protein in the sample. It should be noted that a vast majority of microdialysis samples are analyzed using liquid chromatographic techniques.

Several different approaches for enhancing microdialysis RR that are amenable for HPLC detection have been reported in the literature. These methods include lipo-microdialysis (Kurosaki et al., Biol. Pharm. Bull. 21:194-96 (1998)), cyclodextrin enhancement (Khramov et al., Anal. Chem. 71:1257-64 (1999)), and solid-support enhancement (Petersson et al., Acta Biochim. Pol. 48:1117-19 (2001)). Enhancement approaches for microdialysis sampling that allow direct injection onto an HPLC would be universal with respect to utility with hydrophobic analytes.

Although these approaches enhance the mass transport of small molecules through the microdialysis probe, their extent of enhancement is actually quite modest and is typically 2 to 3 times that for controls. This is most likely due to the binding constant value between the trap and the target analyte. For example, the binding constant between most analytes and cyclodextrins generally ranges between 10 and 1000 M⁻¹ (Rekharsky et al., Chem Rev. 98:1875-1917 (1998)).

The present invention is directed to overcoming these deficiencies in the art.

### SUMMARY OF THE INVENTION

One aspect of the present invention is directed to a method for detection of the presence of one or more target molecules in a sample. This method involves providing a microdialysis unit having a membrane which defines within it a dialysate-containing region and contacting the microdialysis unit with a sample, potentially containing one or more target molecules, under conditions effective to permit molecules in the sample to permeate the membrane and enter the dialysate-containing region as a dialysate. A perfusate liquid comprising capture agents which are specific to the one or more target molecules and are immobilized to solid structures is provided and contacted with the dialysate (desirably within the dialysate-containing region) under conditions effective to permit target molecules present in the dialysate, if any, to specifically bind to the capture agents immobilized to the solid structures. As a result, a dialysate liquid potentially comprising dialysate complexes of target molecules bound to capture agents, which are immobilized to solid structures, is formed. The dialysate liquid potentially is analyzed for dialysate complexes. As a result, the presence of the one or more target molecules in the sample is detected.

Another aspect of the present invention relates to a kit for detection of a presence of one or more target molecules in a sample. The kit comprises a microdialysis unit having a membrane, capture agents that are specific to the one or more target molecules and are immobilized to solid structures, and labeling agents. The labeling agents are suitable for labeling either: (1) any of the target molecules with a label specific for a particular target molecule or (2) the capture agents which are specific to one or more target molecules immobilized to the solid structures.

Using a trapping agent such as an antibody with a much higher binding constant value (10⁶ to 10⁸ M⁻¹) has the possibility of greatly enhancing analyte transport across the microdialysis membrane. The use of antibody-coated microspheres during microdialysis sampling greatly enhances analyte mass transport through the microdialysis membrane. The advantage of using the microspheres is that they are readily amenable for flow cytometric analysis thus allowing a rapid batch sampling process to occur.

### BRIEF DESCRIPTION OF THE DRAWINGS

FIG. 1 is a perspective view of a microdialysis unit in a cannula form.

FIG. 2 is a perspective view of a microdialysis unit in a linear form.

FIG. 3A-C are schematic views of a microdialysis unit depicting the sequential use of the present invention.

FIG. 4 is a schematic view of a system for carrying out the method of the present invention.

FIGS. 5A-D are schematic views of a procedure for carrying out a sandwich assay in accordance with the present invention.

FIGS. 6A-D are schematic views of a procedure for carrying out a competitive assay in accordance with the present invention.

FIG. 7 shows a typical electrospray mass spectrometry experiment. The mass analyzer is a time-of-flight (TOF) analyzer.

### DETAILED DESCRIPTION OF THE INVENTION

One aspect of the present invention is directed to a method for detection of a presence of one or more target molecules in a sample. This method involves providing a microdialysis unit having a membrane which defines within it a dialysate-containing region and contacting the microdialysis unit with a sample, potentially containing one or more target molecules, under conditions effective to permit molecules in the sample to permeate the membrane and enter the dialysate-containing region as a dialysate. A perfusate liquid comprising capture agents which are specific to the one or more target molecules and are immobilized to solid structures is provided and contacted with the dialysate (desirably within the dialysate-containing region) under conditions effective to permit target molecules present in the dialysate, if any, to specifically bind to the capture agents immobilized to the solid structures. As a result, a dialysate liquid potentially comprising dialysate complexes of target molecules bound to capture agents, which are immobilized to solid structures, is formed. The dialysate liquid is analyzed for dialysate complexes. As a result, the presence of the one or more target molecules in the sample is detected.

Another aspect of the present invention relates to a kit for detection of a presence of one or more target molecules in a sample. The kit comprises a microdialysis unit having a membrane, capture agents that are specific to the one or more target molecules and are immobilized to solid structures, and labeling agents. The labeling agents are suitable for labeling either: (1) any of the target molecules with a label specific for a particular target molecule or (2) the capture agents which are specific to one or more target molecules immobilized to the solid structures.

In carrying out the present invention, it is particularly desirable to detect target molecules that are biomolecules. If the target molecules are antigens, then the capture agents comprise antibodies specific to each of the antigens. Should the target molecules be antibodies, the capture agents comprise antigens specific to each of the antibodies. If the target molecules are nucleic acid molecules, then the capture agents comprise nucleic acid molecules complementary to the target molecules. Other examples of biomolecules that can be detected as target molecules, in accordance with the present invention, include cytokines, chemokines, eicosanoids, prostaglandins, leukotrienes, or peptides. Other target molecules that can be detected in accordance with the present invention are drugs, drug metabolites, or pharmacophores.

Microdialysis involves the insertion of a microdialysis probe into a selected tissue or (body) fluid. The probe consists of a small semi-permeable hollow fiber membrane, connected to an inlet and outlet tubing with a small diameter. The probe is continuously perfused with a physiological solution, known as the perfusate. The perfusate is an aqueous solution that must closely match the (ionic) composition of the (extracellular) fluid surrounding the probe in order to prevent unwanted changes in composition and volume of fluid due to osmotic pressure differences. Molecules able to pass the semi-permeable membrane will diffuse across the membrane down their concentration gradient into or out of the perfusate. The solution that exits the probe, the dialysate, can be collected for analysis. Any analytical technique can be used for microdialysate samples as long as it is able to deal with the typical small sample volumes (˜1 to 25 μl) and often low concentrations. The concentrations of the analyte in the dialysate reflect the concentrations in the (extracellular) fluid around the semi-permeable part of the probe. However, as the dialysis procedure is not performed under equilibrium conditions, the concentration in the dialysate will be different from that in the periprobe fluid. The term “recovery” is used to describe this relationship and should be determined by a suitable method for quantification of microdialysis data as described in paragraph 004 above. The solution that exits the dialysate is collected for analysis. Microdialysis systems are described in deLange, et al., “Methodological Issues in Microdialysis Sampling for Pharmacokinetic Studies,” Adv. Drug Deliv. Rev. 45:125-28 (2000); Davies, et al., “Analytical Considerations for Microdialysis Sampling,” Adv. Drug Deliv. Rev. 45:169-88 (2000); Sjogren, et al., “Technical Prerequisites for In Vivo Microdialysis Determination of Interleukin-6 in Human Dermis,” Brit J. Dermatol. 146:375-82 (2002); Winter, et al., “A Microdialysis Method for the Recovery of IL-1B, IL-6, and Nerve Growth Factor from Human Brain In Vivo,” J. Neurosci. Methods, 119:45-50 (2002), which are hereby incorporated by reference in their entirety.

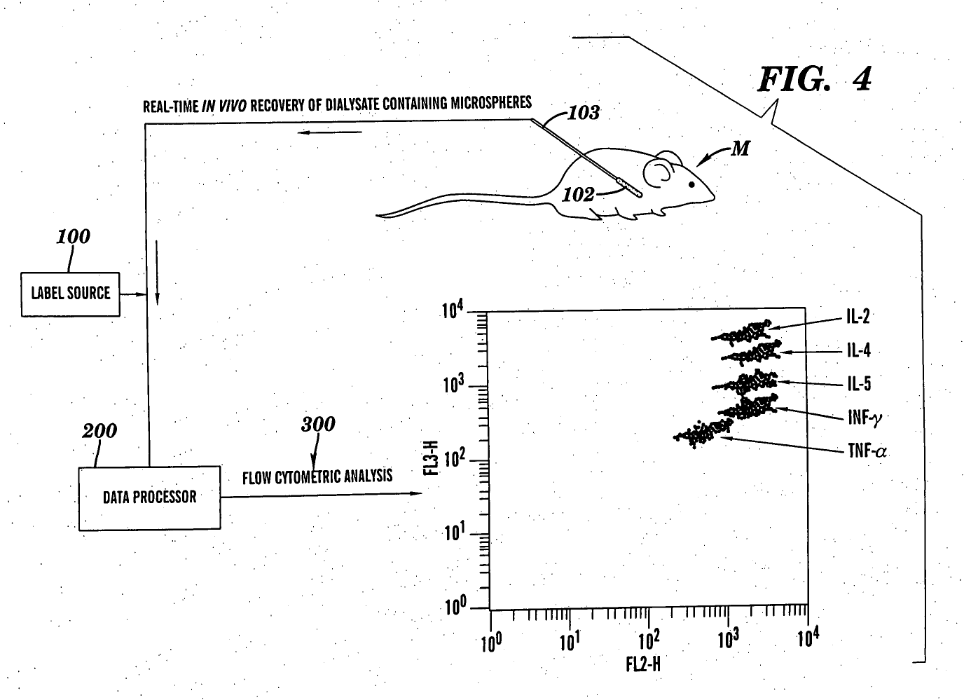

The method of the present invention can be carried out by contacting the microdialysis unit with a sample in vivo. See FIG. 4.

Alternatively, the method of the present invention is carried out by contacting the microdialysis unit with a sample ex vivo. In this embodiment, the microdialysis unit can be placed within an explanted tissue or organ or used within a tissue culture model system.

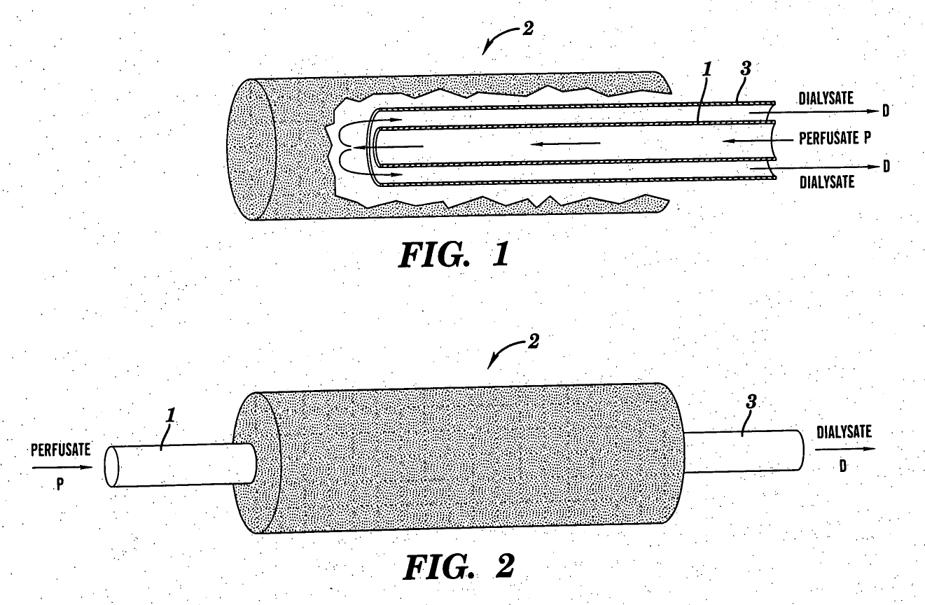

FIG. 1 shows a microdialysis unit that has a cannula form. In this embodiment of the present invention, perfusate P enters microdialysis unit 2 through inlet conduit 1, while dialysate D exits from another side of microdialysis unit 2 through conduit 3.

FIG. 2 shows a microdialysis unit that has a linear form. In this embodiment of the present invention, perfusate P enters microdialysis unit 2 through inlet conduit 1, while dialysate D exits through conduit 3 that concentrically surrounds inlet 1.

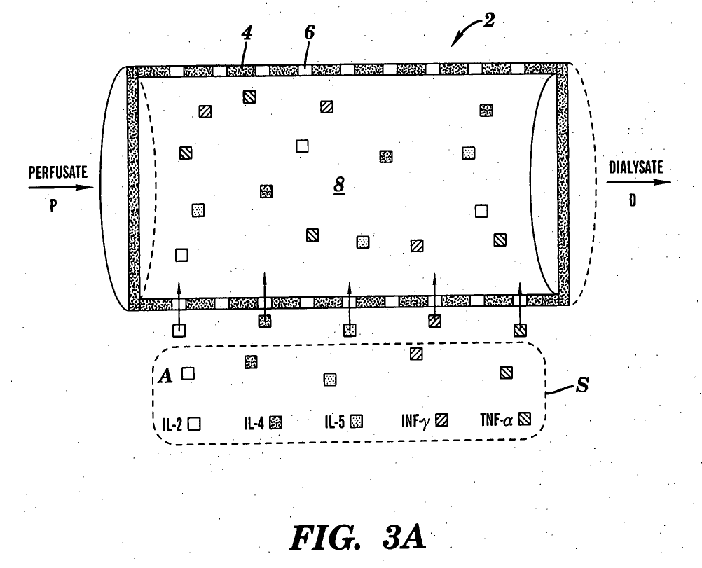

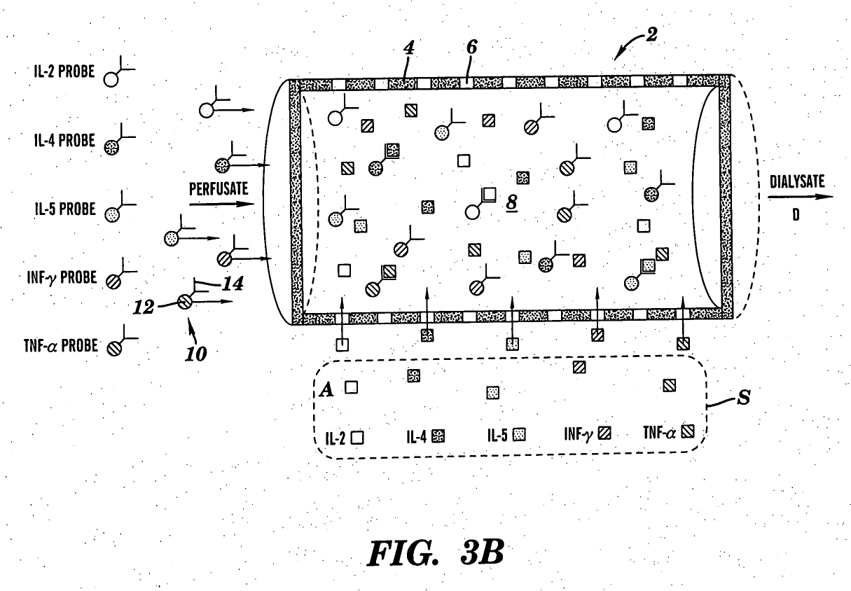

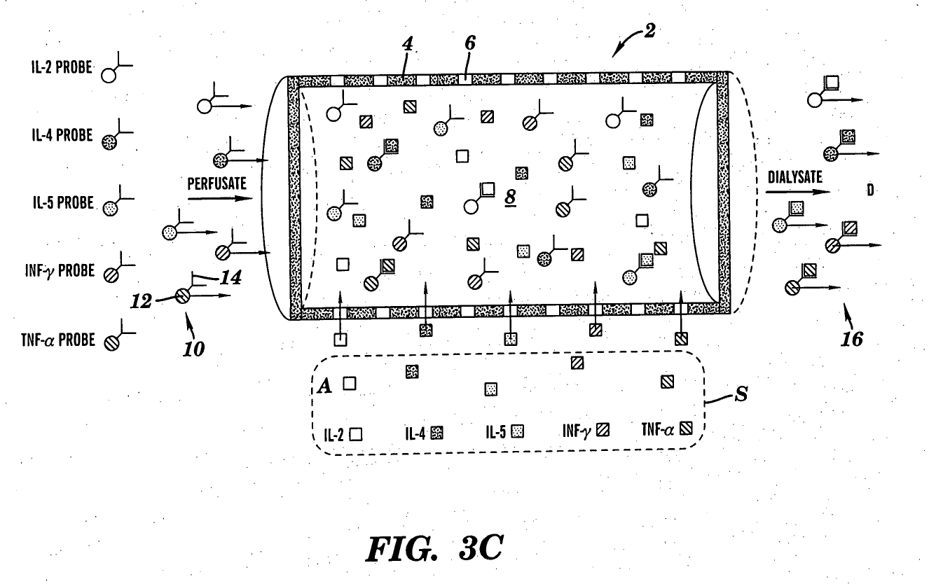

FIGS. 3A-C are schematic views of a microdialysis unit and its sequence of use in accordance with the present invention.

FIG. 3A shows the initial step of this method where the microdialysis unit, receiving perfusate P and discharging dialysate D, is contacted with sample S. Sample S potentially contains antigens A. Once microdialysis unit 2 is contacted with sample S, antigens A will then pass through openings 6 in membrane 2 and enter interior 8 of microdialysis unit 2. Here, sample S happens to include the following antigens A: IL-2, IL-4, IL-5, INF-γ, and TNF-α.

Next, as shown in FIG. 3B, probes 10 which are formed from microspheres 12 to which antibodies 14 specific for the desired antigen are attached, pass with perfusate P to interior 8 of microdialysis unit 2. In this location, antigen and antibodies specific to the antigen have sufficient residence time to permit immunological binding between them. In the case of a nucleic acid target and a complementary nucleic acid capture agent, binding between them involves a hybridization reaction. Microspheres 12 which form probe 10 have hatched shading to indicate that they include or encompass a label and that label is different for each probe and corresponds to a different antigen.

FIG. 3C shows that after the events of FIG. 3B have taken place for a sufficient time, dialysate D containing probe-antigen complexes 16 exit from microdialysis unit 2.

Solid structures that are useful in accordance with the present invention include microspheres and nanocyrstals. The solid structures other than nanocrystals are desirably labeled, preferably with nanocrystals, chromophores, fluorescent moieties, dyes, phosphorescent groups, or chemiluminescent moieties.

The size of nanocrystals (ranging from 5 to 75 atoms in diameter) dictates their fluorescence emission wavelength following excitation via an energy source such as U.V. light, a halogen lamp, or an argon laser (found on most standard flow cytometers). Recently, antibodies have been directly attached to nanocrystals or to microspheres impregnated with nanocrystals, thus providing a multiplex approach to quantitation of analytes (Goldman et al., “Avidin: a Natural Bridge for Quantum Dot-Antibody Conjugates,” J. Am. Chem. Soc., 124:6378-82 (2002), which is hereby incorporated by reference in its entirety). There are many advantages to using nanocrystals rather than microspheres impregnated with traditional fluorophores. Microspheres impregnated with traditional fluorophores are limited by the inability to incorporate more than two or three fluorophores, broad and asymmetrical emission profiles, and spectral overlap. These unfavorable properties limit the technology to only 99 spectrally discernable codes. In contrast, nanocrystals possess superior absorption and emission properties, high stability against photobleaching, and “tunability” which allows for approximately 10,000 to 40,000 spectrally discernable codes. These features make them ideal for incorporation into massively parallel and high-throughput biomolecular analysis platforms. Furthermore, a wide variety of commercially available or non-commercial antibodies can be attached to nanocrystals using simple chemical conjugation techniques (e.g., avidin-biotin binding). This inherent flexibility, which is lacking in most commercial antibody-coated microsphere assay kits, will allow investigators to “tailor” the use of the present invention to their specific drug manufacture and testing, clinical diagnostic, basic research, and military needs.

It is particularly desirable for the label to be coupled to a detection molecule specific for a particular target molecule. In general, the labels for different target molecules can be the same type of label but differ from one another in intensity. Alternatively, the labels for different target molecules can be different types of labels. Useful labels include nanocrystals, chromophores, fluorescent moieties, dyes, phosphorescent groups, or chemiluminescent moieties.

It is particularly desirable for the target molecules detected in the sample to be quantified. As shown in FIG. 4, this can be carried out by flow cytometry. As depicted, microdialysis unit 102 is implanted in mouse M with outlet conduit 103 being arranged to carry dialysate to data processor 200 and flow cytometer 300. Prior to reaching data processor 200 (and, perhaps, before the dialysate leaves interior 8 of microdialysis unit 2), the dialysate is contacted with a labeling agent from label source 100 which, as mentioned previously, can be in a form suitable for labeling either: (1) any of the target molecules with a label specific for a particular target molecule or (2) the capture agents which are specific to one or more target molecules immobilized to the microspheres. Which of these alternatives should be selected depends on the format of the assay that is to be used. Alternative (1) is for a sandwich assay format, while alternative (2) is for a competitive assay format.

The graph in FIG. 4 would be generated as a result of the analysis conducted with this system. By use of particular labels for microspheres attached to the different antibodies specific to different antigens, this graphical representation shows distinctly labeled clusters of data points for different target antigens. The y-axis of this graph is for label intensity with higher values constituting higher intensities. Thus, each target antigen can be distinguished in this embodiment of the present invention with labels of different intensity. The x-axis is a measure of the quantity of labeled microspheres. As a result, the farther to the right a cluster of label points is, the greater the amount of the corresponding target antigen present in the sample being tested.

The flow cytometer is a specialized instrument that can detect fluorescence of individual molecules in a suspension and thereby determine the number of molecules to which a fluorescent probe is bound. The amount of population present is measured by passing the solid structures one at a time through a fluorimeter with a laser-generated incident beam. See Abbas, et al., Cellular and Molecular Immunology, pp. 525-28 (5^{th} ed. 2003), which is hereby incorporated by reference in its entirety. Particle-based flow cytometry procedures involve using microparticles as solid supports for conventional assays with the microparticles being analyzed by passing them through a flow cytometer. Such techniques have been viewed as a solution to the difficulties encountered with other techniques for quantifying soluble analytes. Particle-based flow cytometry is described in more detail in Vignali, “Multiplexed Particle-Based Flow Cytometric Assays,” J. Immunol. Methods. 243:243-55 (2000), which is hereby incorporated by reference in its entirety.

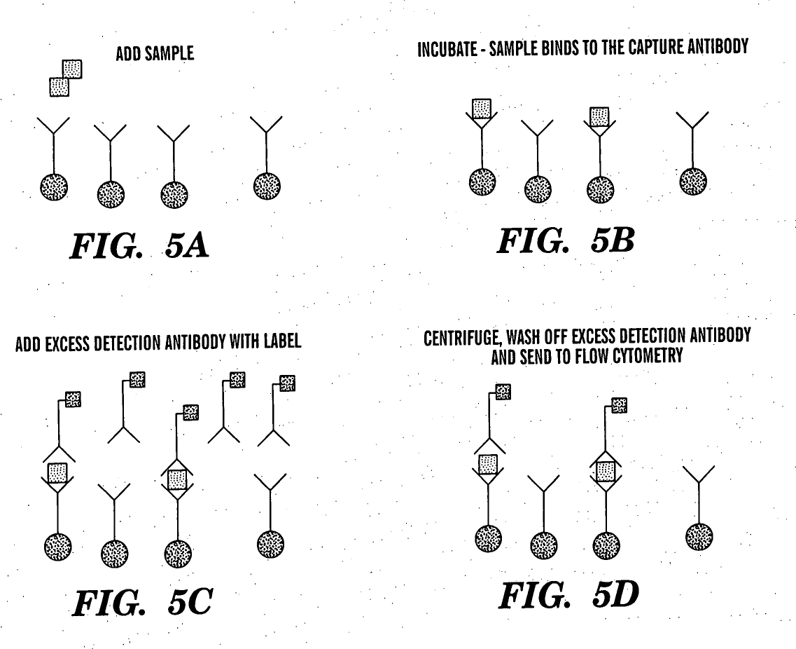

The flow cytometry method for cytokine analysis can be carried out with an immunoassay in a sandwich assay format. The principle behind a sandwich assay is described with reference to FIGS. 5A-D. Most immunoassays use a solid support approach to anchor the capture antibody to make the washing steps easier. Sandwich assays are very commonly applied to the detection of proteins larger than 10,000 Da using an immunoassay. The reason for this is that in the sandwich assay two separate antibodies are required to bind to the target analyte. Since antibodies are large macromolecules, the analyte has to be large enough to prevent steric hindrance during the binding steps. In this format, a sample to be analyzed is combined with immobilized capture antibodies specific for target antigens in the sample and the mixture is permitted to incubate, as shown in FIG. 5A-B. A labeled detection antibody is then added to the mixture, as depicted in FIG. 5C. The mixture is then centrifuged, excess detection antibody is washed away, and the centrifuged and washed material is ready for analysis by flow cytometer 200. See FIG. 5D.

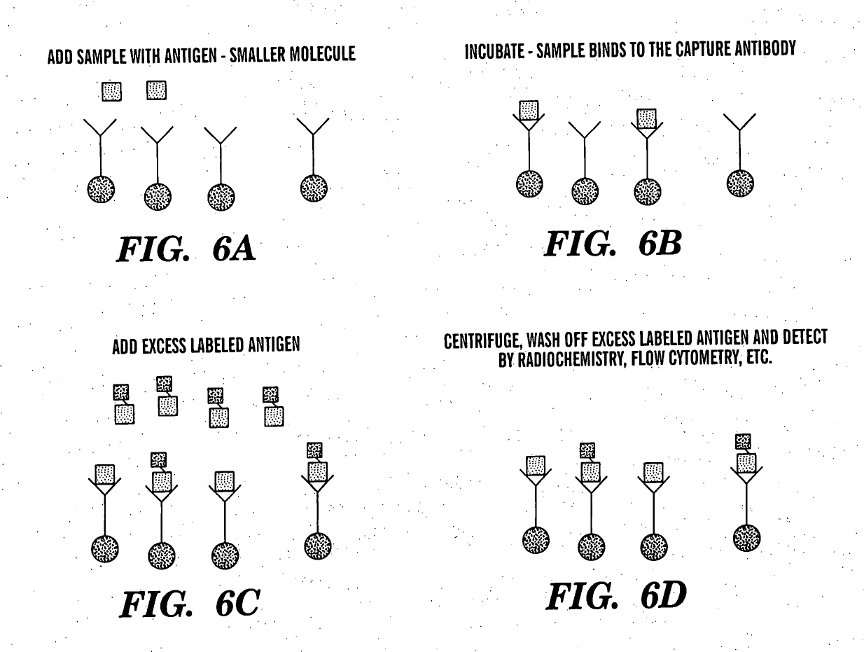

Drugs and small peptides are too small to be analyzed via a sandwich assay. However, competitive assays can be used. In these assays, an excess of labeled antigen, rather than labeled detection antibody is added. In this assay, the concentration of the analyte is inversely proportional to the amount of labeled antigen detected as shown in FIG. 6A-D. In this format, a sample to be analyzed is combined with immobilized capture antibodies specific for target antigens in the sample and the mixture is permitted to incubate, as shown in FIG. 6A-B. A labeled form of the antigen is then added to the mixture, as depicted in FIG. 6C. The mixture is then centrifuged, excess labeled antigen is washed away, and the centrifuged and washed material is ready for analysis by flow cytometer 200. See FIG. 6D.

Through the use of multiple labels that can discriminate between different target molecules, the present invention can be used to detect a plurality of target molecules in a sample.

In one embodiment, the labels for the solid structures and the labels coupled directly or indirectly to the capture agents and the solid structures are the same type of label. However, the labels coupled directly or indirectly to the capture agents and the solid structures differ from one another, for the different target molecules in the dialysate complex, in intensity. This allows for quantitation of the target molecules present in the sample, while the labels of the solid structure serve to discriminate between different target molecules.

In another embodiment of the present invention, the labels for the solid structures are the same type of label and the labels coupled directly or indirectly to the capture agents and the solid structures are different types of labels. This allows for quantitation of the target molecules present in the sample, while the labels of the solid structure serve to discriminate between different target molecules.

The method of the present invention can be used to detect or monitor disease states via chemical analysis of different biomolecules and their expression patterns. For example, pathogens like bacteria, viruses, fungi, and parasites can be identified by virtue of the specific pattern of biological response modifiers (i.e. cytokines, chemokines, eicosanoids, prostaglandins, leukotrienes or peptides) they elicit in their host. In addition, a wide variety of disease states whose origin is non-infectious, inflammatory, hematological, neurological, social stress, eating, autoimmune, allergic, immune efficiency, cancer, etc. can be identified on the basis of specific patterns of biological response modifiers.

Alternatively, the method of the present invention can be used to monitor the progress of treating a subject with a pharmaceutical agent. A wide variety of disease states whose origin is non-infectious, infectious, inflammatory, hematological, neurological, social stress, eating, autoimmune, allergic, immune deficiency, cancer, etc. can be treated with such a pharmaceutical agent to effect a return to base line levels of biological response modifiers. A return to base-line levels would be indicative of full recovery from the disease state.

In another embodiment, the method of the present invention can be used to screen candidate compounds for therapeutic efficacy in treating a subject for a particular condition. For example, this method can be used to monitor the ability of candidate compounds to alter the levels of biological response modifiers that are either elevated or suppressed as a consequence of a disease state like those described in the preceding paragraph.

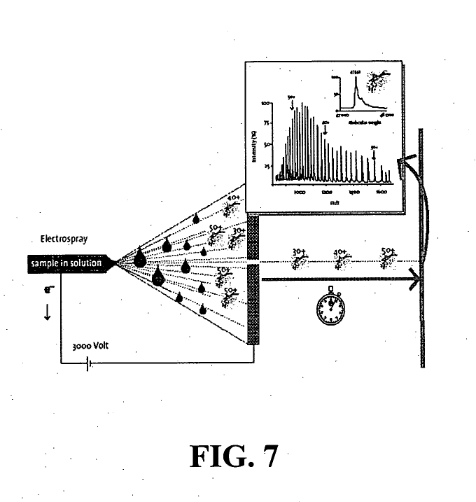

An additional analytical method that would provide great multiplexing benefit would be to couple the relative recovery enhancement using antibodies or antibody-coated beads with mass spectrometry. Mass spectrometry (FIG. 7) is unique among the repertoire of tools available to solve chemical problems in that all molecules have “mass.” The only limitation to mass spectrometry for analysis of any particular analyte would be in the inability to ionize the molecule. However, most biological molecules, especially proteins and peptides can be ionized through a standard electrospray ionization (ESI) source. Mass spectrometry (MS) can be used to provide both sensitive and selective detection capabilities particularly for neuropeptide analysis. Multiplexed neuropeptide analysis is just as important as cytokine analysis, because like cytokines, neuropeptide networks are connected and thus multiple neuropeptides send a message rather than one particular neuropeptide.

For neuropeptide analysis, different antibodies or antibody-coated beads could be simultaneously passed through the microdialysis probe. Free antibodies would most likely be biotinylated to allow capture onto either streptavidin coated beads or plates prior to mass spectrometric analysis. Antigen/antibodies complexes captured onto streptavidin-coated beads could be preconcentrated via centrifugation prior to MS analysis. Alternatively, one could envision a microfluidic platform that would automate the preconcentration process. The advantage of mass spectrometry is that liquid chromatography-mass spectrometry (LC-MS) could be used to separate out the different peptides prior to MS detection. Thus, the LC part provides the much needed selectivity for the multiplexing part of the analysis. The LC part complements the MS since the LC will separate the peptides and the MS will confirm the mass. If components co-elute, the MS can be used to determine the extent of co-elution.

A distinct advantage of using mass spectrometry over the ELISA-based detection schemes is that many antibodies can have cross-reactivity with other analytes. One example is insulin and insulin growth factor (IGF). Both of these analytes bind an insulin antibody and cross react. Thus, gaining quantitative information about the concentrations of both is not possible using standard ELISA technology. Because both of these proteins have different masses, they could easily be picked up by one antibody and then quantified using LC-MS approaches.

### EXAMPLES

### Example 1

Antibody-labeled Microspheres

A BD Pharmingen Mouse Th1/Th2 kit consisting of antibody-labeled microspheres with antibodies against IL-2, IL-4, IL-5, IFN-gamma, and TNF-alpha was used for these experiments.

### Example 2

Microdialysis Sampling

Microdialysis probes (CMA-20) with a 10 mm polyethersulfone membrane (100 kDa MWCO) were purchased from CMA Microdialysis (Acton Mass.) and were used as received. For control experiments, the perfusion fluid contained the assay diluent from the BD Pharmingen kit. To enhance the transport of cytokines during the microdialysis sampling process, 5 μl of bead solution for each target analyte was combined and diluted to 250 μl with the assay diluent. This solution was then passed through the microdialysis probe at a flow rate of 2.0 μl/min using a CMA-102 perfusion pump (CMA Microdialysis, Acton Mass.). Greater than 200 μl of fluid was allowed to pass through the microdialysis probe that was immersed into a standard containing 5000 pg/ml of IL-2, IL-4, IL-5, IFN-gamma, and TNF-alpha. After sample collection, the dialysates containing microspheres were reconstituted in 25 μl of assay diluent. Microspheres with coupled target analyte were then incubated with 25 μl phycoerythrin detection reagent for 2 hours and washed briefly prior to flow cytometric analysis.

### Example 3

Flow Cytometry

The dialysate samples were analyzed using a BD FACScan flow cytometer with the appropriate BD Biosciences software for this analysis.

Different cytokines (e.g., mouse IL-2 (15 kD), IL-4 (20-25 kD), and TNF-α (51 kD, a 17 kD trimer)) have been successfully measured across microdialysis probes (CMA-20, 10 mm, polyethersulfone membrane, MWCO 100 kD) in a quiescent solution using a multiplexed cytokine bead array assay (BD Pharmingen). TNF-α has the largest molecular weight of the cytokines targeted for analysis in this example (Thorpe et al., “Detection and Measurement of Cytokines,” Blood Rev. 6:133-48 (1992), which is hereby incorporated by reference in its entirety). Only 25 μl of sample was needed to simultaneously measure all three of these cytokines in solution using the multiplexed bead array (Carson et al., “Simultaneous Quantitation of 15 Cytokines Using a Multiplexed Flow Cytometric Assay,” J. Immunol. Methods 227:41-52 (1999), which is hereby incorporated by reference in its entirety). Using a flow rate of 1.5 μl/min, the microdialysis E_{d} from a phosphate buffer (pH 7.4) containing 5000 pg/ml cytokines was 1.5±0.5% (TNF-α, n=5), 1.1±0.3% (IL-2, n=3), and 0.9±0.3% (IL-4, n=4). Only 25 μl of dialysate was needed to make these measurements with the microspheres. In an attempt to greatly enhance the cytokine recovery, the microspheres were included in the perfusion fluid as described in Example 2 and the flow rate was set to 2 μl/min for 100 minutes (a typical time used for collecting enough sample for a standard ELISA. The probe was placed into the same phosphate buffer solution containing cytokines at a concentration of 5000 pg/ml. The collected dialysate was centrifuged to allow removal and reconstitution of the beads for detection of cytokines. The cytokine relative recoveries were 35.8%, 16.1%, and 46.9% for TNF-α, IL-2, and IL-4 (n=1), respectively.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for detection of a presence of one or more target molecules in a sample, said method comprising:
providing a microdialysis unit having a membrane which defines within it a dialysate-containing region;
contacting the microdialysis unit with a sample, potentially containing one or more target molecules, under conditions effective to permit molecules in the sample to permeate the membrane and enter the dialysate-containing region as a dialysate;
providing a perfusate liquid comprising capture agents which are specific to the one or more target molecules and are immobilized to solid structures;
contacting the perfusate liquid with the dialysate under conditions effective to permit target molecules present in the dialysate, if any, to specifically bind to the capture agents immobilized to the solid structures, thereby forming a dialysate liquid potentially comprising dialysate complexes of target molecules bound to capture agents which are immobilized to solid structures; and
analyzing the dialysate liquid for dialysate complexes, thereby detecting the presence of the one or more target molecules in the sample.

2. The method according to claim 1, wherein the one or more target molecules are biomolecules.

3. The method according to claim 2, wherein the one or more target molecules are antigens and the capture agents comprise antibodies or binding portions thereof specific to each of the antigens.

4. The method according to claim 2, wherein the one or more target molecules are antibodies and the capture agents comprise antigens specific to each of the antibodies.

5. The method according to claim 2, wherein the one or more target molecules are nucleic acid molecules and the capture agents comprise nucleic acid molecules complementary to the target molecules.

6. The method according to claim 2, wherein the biomolecule is selected from the group consisting of cytokines, chemokines, eicosanoids, prostaglandins, leukotrienes, and peptides.

7. The method according to claim 1, wherein the target molecules are drugs, drug metabolites, or pharmacophores.

8. The method according to claim 1, wherein said contacting the microdialysis unit with a sample is carried out ex vivo.

9. The method according to claim 1, wherein said contacting the microdialysis unit with a sample is carried out in vivo.

10. The method according to claim 1, wherein the microdialysis unit has a linear form.

11. The method according to claim 1, wherein the microdialysis unit has a cannula form.

12. The method according to claim 1, wherein the solid structures are selected from the group consisting of microspheres and nanocrystals.

13. The method according to claim 1, wherein said analyzing is carried out by detecting a label which is coupled directly or indirectly to the capture agents and the solid structures.

14. The method according to claim 13, wherein the labels for different target molecules are the same type of label but differ from one another in intensity.

15. The method according to claim 14, wherein the labels for different target molecules are different types of labels.

16. The method according to claim 15, wherein the labels are selected from the group consisting of nanocrystals, chromophores, fluorescent moieties, dyes, phosphorescent groups, and chemiluminescent moieties.

17. The method according to claim 16, wherein the solid structures are labeled.

18. The method according to claim 17, wherein the labels for the solid structures and the labels coupled directly or indirectly to the capture agents and the solid structures are the same type of label but the labels coupled directly or indirectly to the capture agents and the solid structures differ from one another, for the different target molecules, in intensity to allow for quantitation of the target molecules present in the sample, while the labels of the solid structure serve to discriminate between different target molecules.

19. The method according to claim 18, wherein the labels are selected from the group consisting of nanocrystals, chromophores, fluorescent moieties, dyes, phosphorescent groups, and chemiluminescent moieties.

20. The method according to claim 17, wherein the labels for the solid structures are the same type of label and the labels coupled directly or indirectly to the capture agents and the solid structures are different types of labels to allow for quantitation of the target molecules present in the sample, while the labels of the solid structure serve to discriminate between different target molecules.

21. The method according to claim 20, wherein the labels are selected from the group consisting of nanocrystals, chromophores, fluorescent moieties, dyes, phosphorescent groups, and chemiluminescent moieties.

22. The method according to claim 1, wherein said analyzing is carried out with a sandwich assay format, said method further comprising:
contacting the dialysate liquid with a detection molecule specific for a particular target molecule and coupled to a label under conditions effective to form labeled dialysate complexes.

23. The method according to claim 1, wherein said analyzing is carried out with a competitive assay format, said method further comprising:
contacting the dialysate liquid with a labeled target molecule under conditions effective to cause the capture agents immobilized to the solid structures to be bound to the labeled target molecule, thereby permitting indirect detection of dialysate complexes.

24. The method according to claim 1 further comprising:
quantifying the one or more target molecules detected as being present in the sample.

25. The method according to claim 24, wherein said analyzing and said quantitating are carried out by flow cytometry.

26. The method according to claim 1, wherein said analyzing is carried out by flow cytometry.

27. The method according to claim 1, wherein said method is used to detect or monitor a disease state.

28. The method according to claim 27, wherein the disease state is caused by a pathogen selected from the group consisting of bacteria, viruses, fungi, and parasites.

29. The method according to claim 27, wherein the disease state has an origin which is either non-infectious, inflammatory, hematological, neurological, social stress, eating, autoimmune, allergic, immune deficiency, or cancer.

30. The method according to claim 1, wherein said method is used to monitor progress of treating a subject having a disease state with a pharmaceutical agent.

31. The method according to claim 30, wherein the disease state has an origin which is either infectious, non-infectious, inflammatory, hematological, neurological, social stress, eating, autoimmune, allergic, immune deficiency, or cancer.

32. The method according to claim 1, wherein said method is used to screen a candidate compound for therapeutic efficacy in treating a subject for a disease state.

33. The method according to claim 32, wherein said method is used to monitor a candidate compound's ability to alter levels of biological response modifiers that are either elevated or suppressed by the disease state.

34. The method according to claim 32, wherein the disease state has an origin which is either infectious, non-infectious, inflammatory, hematological, neurological, social stress, eating, autoimmune, allergic, immune deficiency, or cancer.

35. The method according to claim 1, wherein the presence of a plurality of target molecules in a sample are detected.

36. The method according to claim 1, wherein said contacting the perfusate liquid with the dialysate is carried out within the dialysate containing region.

37. A kit for detection of a presence of one or more target molecules in a sample, said kit comprising:
a microdialysis unit having a membrane;
capture agents which are specific to the one or more target molecules and are immobilized to solid structures; and
labeling agents for labeling either: (1) any of the target molecules with a label specific for a particular target molecule or (2) the capture agents which are specific to the one or more target molecules and immobilized to the solid structures.

38. The kit according to claim 37, wherein the capture agents are specific for one or more target molecules that are biomolecules.

39. The kit according to claim 37, wherein the one or more target molecules are antigens and the capture agents comprise antibodies or binding portions thereof specific to the antigens.

40. The kit according to claim 38, wherein the one or more target molecules are nucleic acid molecules and the capture agents comprise nucleic acid molecules complementary to the target molecules.

41. The kit according to claim 38, wherein the biomolecule is selected from the group consisting of cytokines, chemokines, eicosanoids, prostaglandins, leukotrienes, and peptides.

42. The kit according to claim 37, wherein the target molecules are selected from the group consisting of drug metabolites, pharmacophores, and drugs.

43. The kit according to claim 37, wherein the microdialysis unit has a linear form.

44. The kit according to claim 37, wherein the microdialysis unit has a cannula form.

45. The kit according to claim 37, wherein the solid structures are selected from the group consisting of microspheres and nanocrystals.

46. The kit according to claim 37, wherein the labels for different target molecules are the same type of label but differ from one another in intensity.

47. The kit according to claim 37, wherein the labels for different target molecules are different types of labels.

48. The kit according to claim 37, wherein the labels are selected from the group consisting of nanocrystals, chromophores, fluorescent moieties, dyes, phosphorescent groups, and chemiluminescent moieties.

49. The kit according to claim 37, wherein the solid structures are labeled.

50. The kit according to claim 49, wherein the labels for the solid structures and the labels of the labeling agents are the same type of label but the labels of the labeling agents differ from one another in intensity so that the labels in the labeling agents allow for quantitation of the target molecules present in the sample, while the labels of the solid structure serve to discriminate between different target molecules.

51. The kit according to claim 50, wherein the labels are selected from the group consisting of nanocrystals, chromophores, fluorescent moieties, dyes, phosphorescent groups, and chemiluminescent moieties.

52. The kit according to claim 49, wherein the labels for the solid structures are the same type of label and the labels of the labeling agents are different types of labels so that the labels of the labeling agents allow for quantitation of the target molecules present in the sample, while the labels of the solid structure serve to discriminate between different target molecules.

53. The kit according to claim 52, wherein the labels are selected from the group consisting of nanocrystals, chromophores, fluorescent moieties, dyes, phosphorescent groups, and chemiluminescent moieties.

54. The kit according to claim 37, wherein the kit is in a sandwich assay format with the labeling agent comprising a detection molecule specific for a particular target molecule and coupled to a label, whereby the labeling agent binds to target molecules bound to the capture agents immobilized to the solid structures.

55. The kit according to claim 37, wherein the kit is in a competitive assay format with the labeling agent comprising a labeled target molecule, whereby labeled target molecule binds to the capture agents immobilized to the solid structures, thereby permitting indirect detection of dialysis complexes formed from target molecules bound to the capture agents.
